(19) United States
(12) Patent Application Publication
(10) Pub. No.: US 2010/0247491 A1
(43) Pub. Date: Sep. 30, 2010

(54) Potentiation of Stem Cell Homing and Treatment of Organ Dysfunction or Organ Failure

(76) Inventor: Christof Westenfelder, Salt Lake City, UT (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/525,592

(22) PCT Filed: Feb. 1, 2008
(86) PCT No.: PCT/US08/01371

§ 371 Date: Jun. 15, 2010

Related U.S. Application Data
(60) Provisional application No. 60/898,803, filed on Feb. 1, 2007.

(51) Int. Cl. A61K 35/12; A61P 13/12

(52) U.S. Cl. 424/93.7

(57) ABSTRACT

The invention provides methods and compositions for the treatment of multi-organ failure or kidney dysfunction, such as acute renal failure, by mesenchymal stem cells and a CD26 inhibitor, where inhibition of CD26 increases homing of the mesenchymal stem cells to a target tissue.

### STATEMENT OF CROSS-RELATED APPLICATIONS

This application claims the benefit of U.S. Provisional Patent Application No. 60/898,803, filed Feb. 1, 2007, the entirety of which is incorporated by reference.

### TECHNICAL FIELD

This invention generally relates to the use of mesenchymal stem cells and derivatives thereof for the treatment of organ dysfunction and/or organ failure.

### BACKGROUND

Stem cell therapies hold significant promise for the development of new treatments for a wide range of diseases. Stem cells are undifferentiated cells capable of self-renewal and differentiation into other cells types, and may be characterized based on cell surface markers, the presence or absence of transcription factors and/or production of cytokines. Bone marrow-derived stem cells include hematopoietic stem cells (HSCs) and Mesenchymal stem cells or marrow stromal cells (MSCs). MSCs have a large capacity for self-renewal and the ability to differentiate into cell types that include chondrocytes, osteocytes, myocytes, endothelial cells, neurons, beta-pancreatic islet cells and adipocytes.

Transplanted allogeneic MSCs have been shown to modulate the immune system of a subject. For example, Aggarwal and Pittenger demonstrated that human MSCs (hMSCs) cocultured with subpopulations of immune cells altered the cytokine secretion profile of dendritic cells (DCs), T cells (TH1 and TH2), and natural killer cells to reduce the inflammatory response. Sudeepta Aggarwal, and Mark F. Pittenger, (2005), Human Mesenchymal Stem Cells Modulate Allogeneic Immune Cell Responses, Blood 105:1815-1822. In addition, administration of MSCs has been found to be beneficial in the treatment of organ dysfunction and organ failure, including, but not limited to, acute renal failure (ARF) and multi-organ failure (see International Patent Publications WO 2004/090112 and WO 2006/121445, the entirety of both are hereby incorporated by reference). This is believed to be due to the ability of MSCs to reach intrarenal sites of injury and provide a beneficial paracrine affect to the organ, for example, reducing an inflammatory response in the kidney. One of the primary effects of ARF on the kidneys is the destruction of kidney tubular and vascular cells. Recovery of kidney function and patient survival depends primarily on the protection and regeneration of the destroyed and injured cells.

While MSCs are capable of reducing the inflammatory response and helping patients recover or survive ARF, the ability to target MSCs to the kidney would be a significant improvement in the art.

### SUMMARY OF THE INVENTION

The invention relates to a method of improving the homing efficiency of administered MSCs and a method of increasing the renal protective effects of MSCs.

The invention also relates to the use of mesenchymal stem cells (which may be syngeneic (which includes autologous) or allogeneic (non-autologous)) for the treatment of organ dysfunction and/or organ failure in a subject. In an exemplary embodiment the invention relates to the use of MSCs for the treatment of multi-organ failure or kidney dysfunction, such as acute renal failure, in combination with the use of a CD26 inhibitor. In another exemplary embodiment the invention relates to the use of a chemotropic agent in combination with MSCs. In yet another exemplary embodiment the invention relates to the use of MSCs in combination with a CD26 inhibitor and/or a chemotropic agent (e.g., SDF-1 or SDF-1 analogue).

In a further exemplary embodiment, the invention relates to a method of treating a subject thought to be suffering from organ dysfunction or organ failure, for example, renal failure or injury, by administering one or more CD26 inhibitors to the subject. In another exemplary embodiment a CD26 inhibitor is administered to a subject in combination with MSCs, for example, either contemporaneously with the MSCs or prior to administration of the MSCs. In yet another exemplary embodiment, the MSCs are pre-treated with a CD26 inhibitor prior to administering the MSCs to a subject.

In another exemplary embodiment the CD26 inhibitor comprises a peptide analog designed by chemical modification of a natural CD26 inhibitor, wherein the chemical modification may comprise: modifications to the N and/or C terminal ends of the peptide (e.g., N terminal acetylation or desamination and/or replacement of the C-terminal carboxyl group with an amide or alcohol); changes to the side chain, which may involve amino acid substitutions; modification of the a carbon including methylations, alkylations and dehydrogenations; replacing one or more D residues with one or more L residues; and introduction of amide bond replacements, i.e., changing the atoms participating in the peptide (or amide) bond.

In another exemplary embodiment the invention relates to the use of a CD26 inhibitor in combination with the administration of MSCs to provide time savings when syngeneic MSCs are desired, as fewer cells need to be grown. In another exemplary embodiment, the invention relates to the use of a CD26 inhibitor in combination with administration of MSCs to reduce the number of MSCs administered to a subject.

The invention also relates to an improved method of treatment for organ dysfunction and/or organ failure, including such diseases as acute renal failure, acute kidney injury, multiple-organ failure, early dysfunction of kidney transplant, graft rejection, chronic renal failure, and similar diseases. In another exemplary embodiment the invention relates to a method of treating a disease condition wherein the damaged organ increases production of SDF-1.

The invention also relates to the manufacture of a medicament comprising a CD26 inhibitor for the treatment of a disease associated with organ dysfunction and/or organ failure. Optionally, the medicament may further comprise MSCs, a chemotropic agent, or combinations thereof.

### BRIEF DESCRIPTION OF THE DRAWINGS

FIGS. 1A and 1B illustrate the affect of acute renal failure on the levels of SDF-1 protein in the kidney, plasma and bone marrow.

FIG. 2 illustrates the effect of a CXCR4 blocking antibody on the migration of CXCR4⁺/CD34⁺MSCs.

FIG. 3 indicates that MSCs are capable of treating acute renal failure (ARF).

FIG. 4 shows that inhibition of CD26 can increase homing of HSCs from the circulation to bone.

FIG. 5 shows that human MSCs express CD26, SDF-1, and CXCR4.

FIG. 6 shows that MSCs express functional CD26.

FIG. 7 shows that inhibition of CD26 significantly increases migration of MSCs.

FIG. 8 illustrates an exemplary experimental outline.

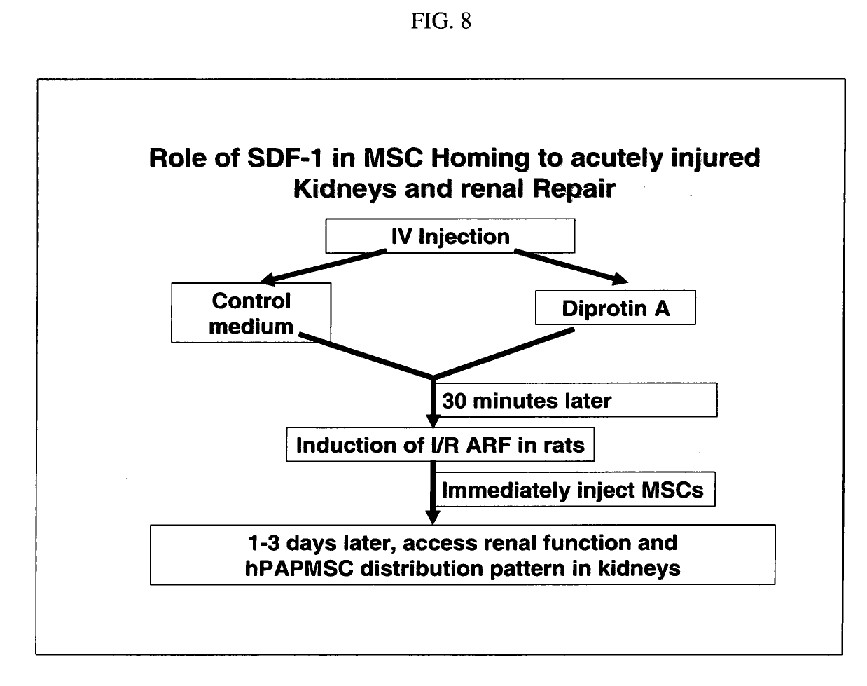

### DETAILED DESCRIPTION OF THE INVENTION

As used herein and in the appended claims, the singular form, for example, “a”, “an”, and “the,” includes the plural, unless the context clearly dictates otherwise. For example, reference to “a CD26 inhibitor” includes a plurality of such inhibitors, such as a mixture of two or more CD26 inhibitors, and equivalents thereof.

As used herein, the phrases “co-administration,” “in combination with,” “the combination of” or similar phrases referring to a compound (I.e., a CD26 inhibitor or chemotropic agent) and MSCs, means that the effect of the compound and MSCs are both present in the subject being treated. The compound and/or MSCs may be administered at the same time or sequentially in any order at different points in time. However, the compound and MSCs should be administered sufficiently closely in time so as to provide the desired enhancement of treatment effect. The compound and MSCs may be administered by the same route of administration or by different routes of administration. Suitable dosing intervals, routes and the order of administration with such compound and MSCs, in light of the present disclosure, will now be apparent to those skilled in the art.

As used herein, a “CD26 inhibitor” means any compound, peptide or peptidomimetic capable of inhibiting CD26 protease activity. Examples of CD26 inhibitors include, but are not limited to, Diprotin A (H-Ile-Pro-Ile-OH), L-Val-L-boroPro, sulphostin, P-epi sulphostin, sulphostin desulfonate, Lys[Z(NO2)]-thiazolidide, Lys[Z(NO2)]-pyrrolidide, LAF237, MK-0431, Sitagliptin (Januvia™), vildagliptin, Saxagliptin, Allogliptin, and/or a salt or ester thereof (see also, U.S. Pat. No. 6,100,234, International Patent Publication 2007/041368 and references cited therein).

As used herein, “MSCs” or “mesenchymal stem cell” means a population of cells wherein the majority of cells in the population retain the ability to self-renew and differentiate into an adipocyte or osteocyte cell type.

As used herein, a “subject” means any animal, including a human.

As used herein, “treatment” means ameliorating at least one symptom of a disease.

MSCs (also known as marrow stromal cells, bone marrow stromal cells, stromal precursor cells, colony forming unit-fibroblasts, and multipotent adult progenitor cells (MAPC)) are adult stem cells that have been classically obtained from the bone marrow, but may be obtained from other tissues as well, including, but not limited to, peripheral blood, skin, hair root, muscle or fat tissue, umbilical cord tissue, and primary cultures of various tissues. MSCs may be obtained, purified and/or identified by a number of art recognized procedures, such as those described in WO 2006/121445, WO 2004/090112, and Togel et al. (2005) Administered Mesenchymal Stem Cells Protect Against Ischemic Acute Renal Failure Through Differentiation-independent Mechanisms, Am. J. Physiol. Renal Physiol. 289:F31-F42.

SDF-1 (also known as CXCL12) is an α-chemokine (chemotropic agent) that has a chemotactic activity through interaction with the CXCR4 receptor (cysteine-X-cysteine receptor 4) for hemopoietic stem cells (HSCs). Christopherson II et al. (2003) Cell Surface Peptidase CD26/DPPIV Mediates G-CSF Mobilization of Mouse Progenitor Cells, Blood 101(12):4680-4686. SDF-1 also plays important roles in developmental neurobiology, inflammation, stem cell traffic to the bone marrow and injured organs such as the brain, heart, liver and kidneys. SDF-1 regulates adhesion of HSCs to the endothelium, expression of matrix metalloproteinases (MMPs) and other processes involved in HSC homing and engraftment. Recently, it has been demonstrated that renal tubular expression and plasma levels of SDF-1 are markedly up-regulated following induction of acute ischemia/reperfusion kidney injury (AKI) in mice. This increase in SDF-1 specifically boosted the homing of CD34 and CXCR4 co-expressing bone marrow stem cells to the kidney. Togel et al. (2005) Renal SDF-1 signals mobilization and homing of CXCR4-positive cells to the kidney after ischemic injury, Kidney Int. 67:1772-1784 and Son et al. (2007) Migration of Bone Marrow and Cord Blood Mesenchymal Stem Cells in vitro is Regulated by SDF-1-CXCR4 and HGF-c-met Axes and Involves Matrix Metalloproteinases, DOI: 10.1634/stemcells.2005-0271.

CD26 (dipeptidylpeptidase IV; EC 3.4.14.5) is a membrane-bound extracellular peptidase that cleaves dipeptides from the N-terminus of polypeptide chains after a proline or alanine residue. CD26 has the ability to cleave stromal cell-derived factor 1 (SDF-1) (also known as CXC ligand 12) at its position-2 proline and thereby inactivate SDF-1.

Furthermore, administration of MSCs protects against acute kidney injury (AKI) through complex paracrine mechanisms (such as anti-inflammatory, anti-apoptotic and mitogenic cellular response) (Tögel et al. Am J Physiol 289: F31-42, 2005). Here it is shown that inhibiting dipeptidylpeptidase IV (CD26), thereby decreasing inactivation of SDF-1 and increasing SDF-1 mediated homing of CXCR4 expressing MSCs to the SDF-1 signal. In particular, this allows for the homing of MSCs to the kidney in response to renal damage, since the SDF-1 signal is increased in an injured kidney. Thus, the invention provides an improved method of targeting MSCs to an injured kidney or other tissue expressing SDF-1, thereby increasing renal protection and repair by MSCs. Further, the invention provides a method of improving SDF-1 based chemokine therapies.

The present invention demonstrates that inhibition of CD26 in vitro enhances SDF-1-CXCR4 mediated chemotactic activity of MSCs. In contrast, inhibiting CXCR4 decreases the SDF-1-CXCR4 mediated chemotactic activity of MSCs. Thus, in vivo pre-treatment of rats with a CD26 inhibitor, such as Diprotin A, may potentiate the renal protective functions of administrated MSCs in experimental acute kidney injury (AKI). As a result, the invention provides for the use of modified CD26 inhibition protocols and a consequent reduction in the number of MSCs that are needed to achieve robust renoprotection in AKI.

Treatment of a subject with a CD26 inhibitor provides a method of further increasing and/or prolonging the intrarenal concentration of SDF-1, thereby increasing homing or recruitment of MSCs. It is believed that administration of one or more CD26 inhibitors, particularly in combination with the administration of MSC, will potentiate homing or recruitment of endogenous MSCs and/or administered MSCs to the injured organ and result in a beneficial treatment.

The dosage for treatment typically depends upon the route of administration, the age, weight and condition of the subject to be treated. Based on the information presented herein, determination of the effective amount of a CD26 inhibitor, chemotropic agent (e.g., SDF-1) and/or MSCs is optimally determined by a skilled physician (see, WO 2006/121445; U.S. Patent Pub. 2004/0247574; and U.S. Pat. Nos. 6,258,597; 6,300,314; and 6,355,614). The CD26 inhibitor, chemotropic agent and/or MSCs of the invention are generally useful in pharmaceutical compositions which may include any desirable carrier, diluent or vehicle.

The CD26 inhibitor of the invention may be administered to a subject at dosage of about 1 to about 100 μM/kg total body weight, or about 1-50 μM/kg total body weight, or about 1-30 μM/kg total body weight, or about 1-10 μM/kg total body weight. MSCs of the invention may be administered to a subject in an amount of about 0.01 to about 1×10⁶ cells per kilogram of total body weight.

A person of ordinary skill in the art may prepare formulations of the compound(s) (e.g., CD26 inhibitor, chemotropic agent, and combinations thereof) and/or MSCs of this invention for storage or administration by mixing the compound(s) and/or MSCs having a desired degree of purity with physiologically acceptable carriers, excipients, stabilizers etc. Acceptable carriers, excipients, stabalizers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and may include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween, Pluronics or polyethyleneglycol. Sterile compound(s) and/or cells for injection can be formulated according to conventional pharmaceutical practice. For example, dissolution or suspension of the compound(s) and/or cells in a vehicle such as phosphate buffered saline. Buffers, preservatives, antioxidants and the like can be incorporated according to accepted pharmaceutical practice.

SDF-1 is a typical chemokine with a β-β-β-α topology that plays important roles in developmental neurobiology, infection, stem cell trafficing to the bone marrow and injured organs such as brain, heart, liver and kidneys, CXCR4 is a G protein-coupled cell surface receptor for SDF-1 that is expressed on many cells, and CD26 is a dipeptidyl peptidase IV that inactivates SDF-1. In addition, CXCR4 is used by the HIV-1 virus as a co-receptor of CD4 (cluster of differentiation 4) to facilitate binding and destroy T lymphocytes. SDF-1 competes with HIV glycoprotein 120 for binding to CXCR4, which may help to block HIV infection. CXCR4 is also expressed in cancer cell lines (pancreas, esophagus, breast), kidney cells, bone marrow, and neurons.

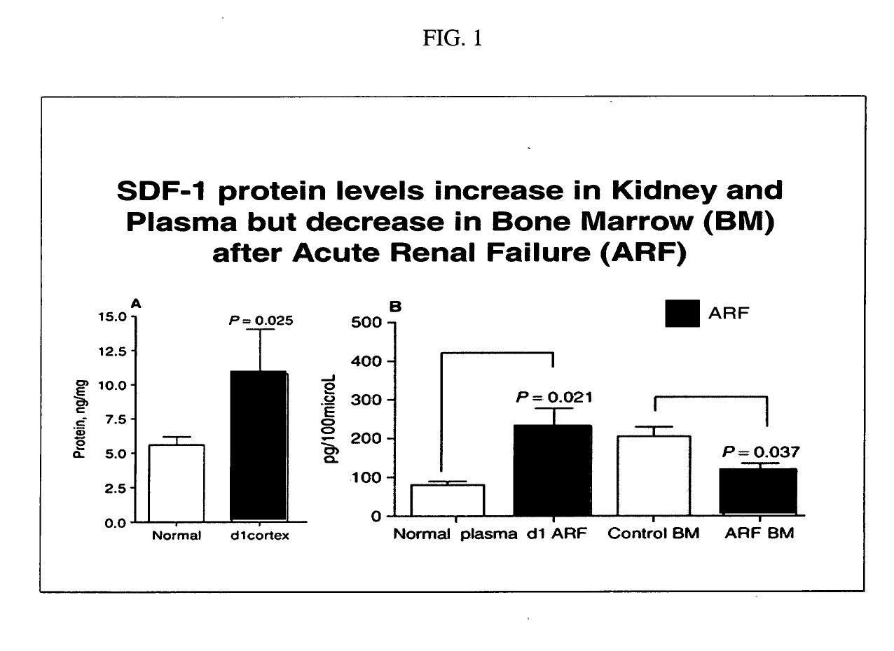

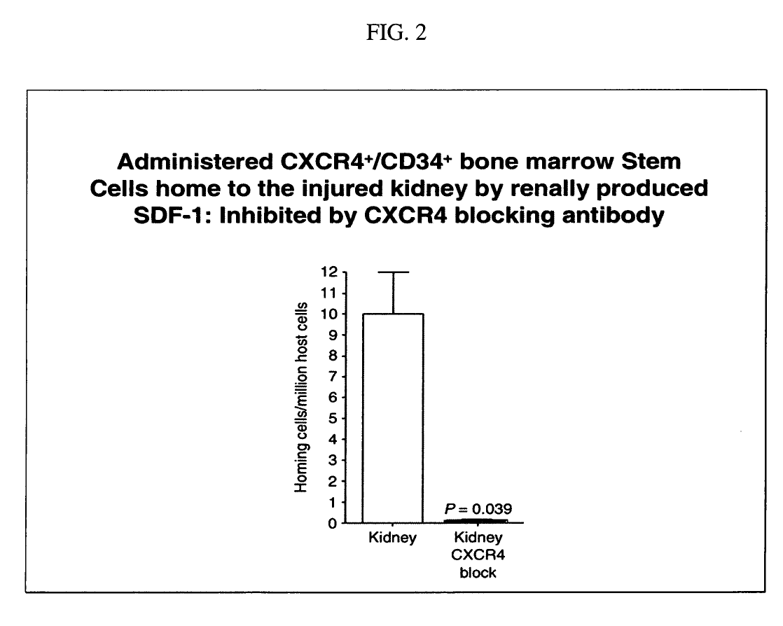

FIGS. 1A and 1B illustrate the affect of acute renal failure on the levels of SDF-1 protein. SDF-1 protein levels in the kidney cortex were significantly elevated at 24 hours after ARF compared to normal kidney levels (FIG. 2A). SDF-1 protein in blood plasma was significantly elevated at day 1 after ARF compared to baseline (FIG. 2B). In contrast, bone marrow (BM) SDF-1 levels were reduced compared to the baseline, thus, reversing the normal bone marrow blood gradient. Togel et al. (2005).

SDF-1 protein quantification in kidneys before (N=3) and 1 day after ARF (N=3), bone marrow, and peripheral blood were carried out by ELISA. Decapsulated kidney tissues were minced, sonicated, lysed with RIPA buffer for cell and tissue lysis and protein quantified by BCA protein assay reagent assay. Bone marrow from femurs of mice (N=3) was flushed with PBS and cells were spun down. Cell fractions and supernatants were analyzed for SDF-1. Equal protein amounts were assayed for SDF-1; 96-well plates were coated with SDF-1 antibody and incubated overnight. Wells were washed with buffer and incubated for 1 hour at room temperature with blocking buffer. One hundred microliters of sample were loaded and incubated for 2 hours at room temperature. After three washing steps, anti-SDF-1 antibody was applied and samples were incubated for 2 hours at room temperature. After washing, horseradish peroxidase conjugate was added to the reaction mixture and incubated for 20 minutes at room temperature. The reaction was stopped and optical density was determined with a microplate reader set at 450 nm. Wavelength correction was set to 570 nm. Sample results were calculated from a standard curve generated by dilutions of a known amount of recombinant SDF-1 protein.

FIG. 2 illustrates the effect of a CXCR4 blocking antibody on the migration of CXCR⁺/CD34⁺MSCs. This experiment indicates that CXCR4 is required for homing of MSCs to an injured kidney. One million bone marrow cells from normal FVB (sensitivity to the B strain of Friend leukemia virus) mice were stained with CFDA (carboxyfluorescein diacetate) and injected into the tail vein after 60 minutes of renal pedicle clamping. Animals were sacrificed at 24 hours and kidneys were obtained for further analysis. One quarter of each kidney was digested with collagenase for 60 minutes at 37° C. After centrifugation and washing with PBS, kidney cells were re-suspended and put into a Neubauer chamber for counting. Injected and homed cells were clearly recognizable by their bright green CFDA staining. CFDA-positive cells as well as total cell numbers were counted in at least two samples of the same kidney and averaged.

Bone marrow was obtained by flushing both femurs with PBS and quantification of homed cells was carried out by counting CFDA-positive cells in relation to bone marrow cells in a Neubauer chamber. To determine the role of CXCR4 in the homing of CXCR4-expressing cells to the injured kidney, bone marrow cells were pre-incubated with 10 ug anti-CXCR4 blocking antibody (eBioscience) for 30 minutes and cells were injected after washing and centrifugation. Kidney and bone marrow were examined for CFDA-positive cells as described above.

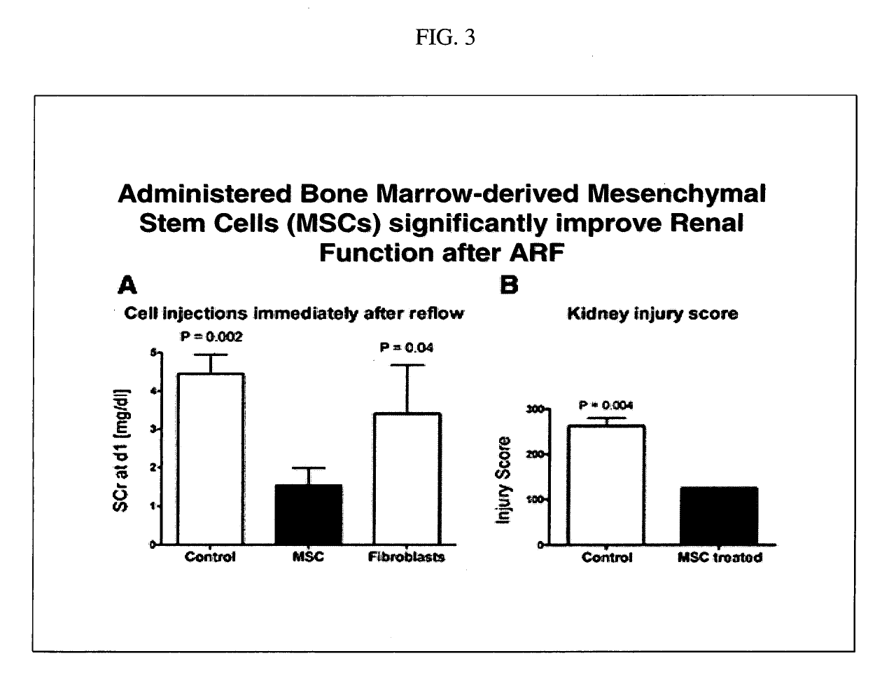

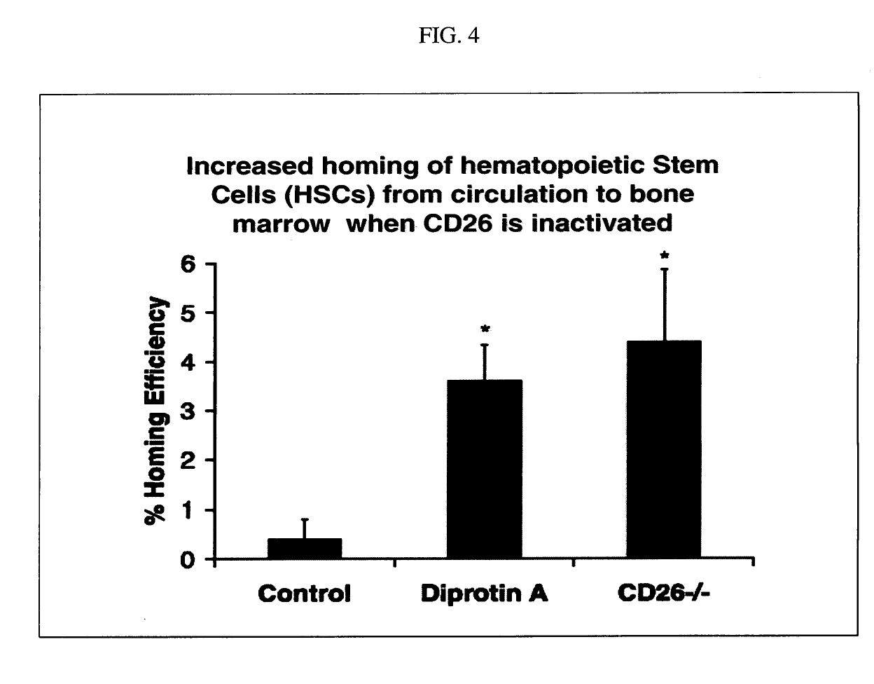

FIG. 3 illustrates the treatment of severe acute renal failure (ARF) with control medium, MSCs or fibroblasts. In FIG. 4A MSCs were administration immediately after reflow to rats with ARF and found to significantly improve renal function at 24 h after clamping. P=0.002, control- vs. MSC-treated animals; P=0.04, fibroblast- vs. MSC-treated animals. P>0.05, vehicle- vs. fibroblast-treated animals. In FIG. 4B MSCs administration is shown to significantly lower kidney injury scores. Creatinine is a breakdown product of creatine, which is an important component of muscle. Creatinine is excreted from the body entirely by the kidneys. With normal renal excretory function, the serum creatinine level should remain constant and normal. Therefore, creatinine levels are frequently used as an indicator of kidney function.

FIG. 4 demonstrates that inhibition of CD26 can increase homing of HSCs from the circulation to bone. Christopherson II et al. (2004). However, the affect of inhibiting CD26 in MSCs remained to be determined. Control, Diprotin

A treated, or CD26−/− sorted Sca-1+lin-donor cells (1-2×10⁴ cells per recipient mouse) were transplanted by tail-vein injection into lethally irradiated female recipient mice. Cells treated with CD26 inhibitors were treated with 5 mM Diprotin A (Ile-Pro-Ile, Peptides International, Louisville, Ky.) for 15 minutes and washed prior to transplant. Percent homing efficiency is calculated by dividing the number of Sca-1+lin-cells in the recipient's BM (2 femurs) by the number of injected Sca-1+lin-donor cells.

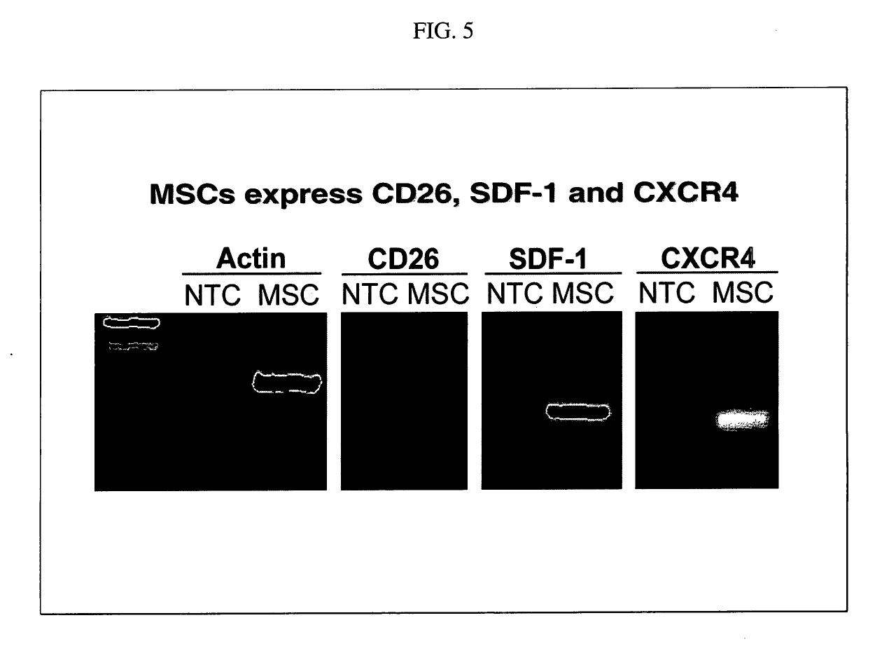

FIG. 5 shows that human MSCs express CD26, SDF-1, and CXCR4. In human MSCs (passage 1-7, CD34−, CD45− and bound to SH2 monoclonal antibody), CXCR4 mRNA levels were too low to be easily detected by routine RT-PCR (Wynn et al. 2004 A Small Proportion of Mesenchymal Stem Cells Strongly Express Functionally Active CXCR4 Receptor Capable of Promoting Migration to Bone Marrow, Blood, 104(9):2643-2645).

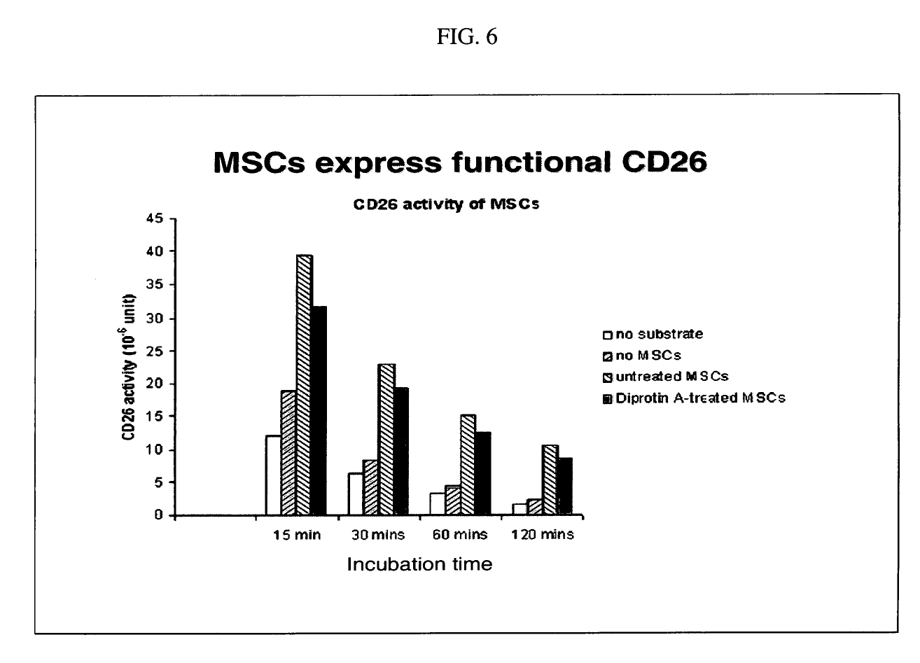

FIG. 6 shows that MSCs express functional CD26. The assay was conducted using Gly-Pro-4-Nitroanilide+H2O which is cleaved by CD26 to produce Gly-Pro+4-Nitroaniline. Using a spectrophotometer, Nitroaniline absorbance at 405 nm was measured and plotted against a standard absorbance curve for Nitroaniline. One unit CD26 produces 1.0 mmole of 4-Nitroaniline from Gly-Pro-4-nitroaniline per minute.

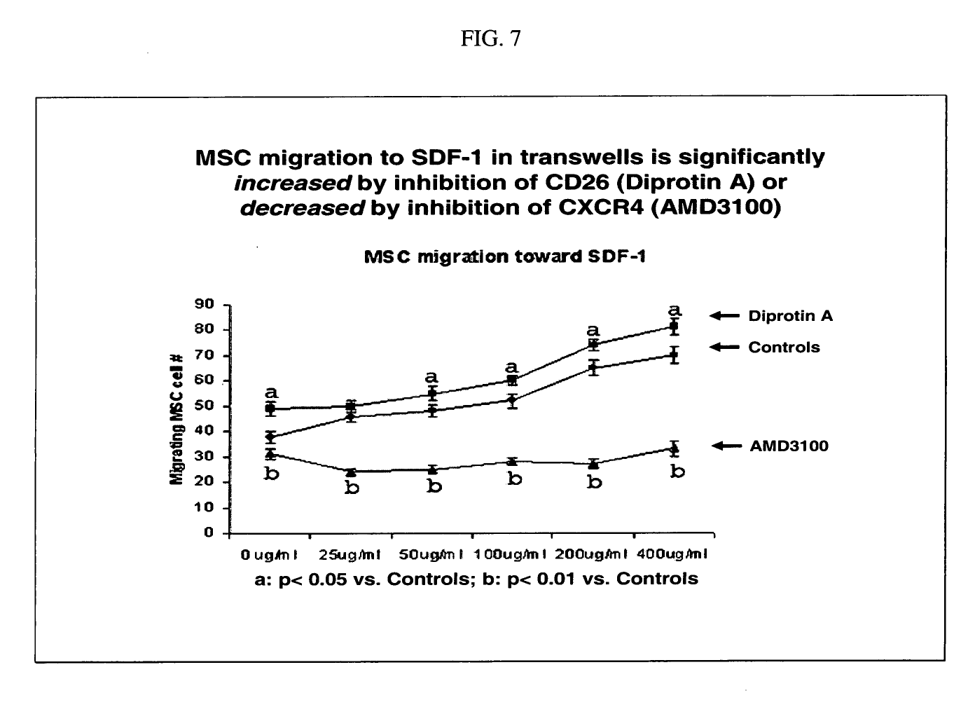

FIG. 7 shows that inhibition of CD26 significantly increases migration of MSCs. AMD 3100 is a non-peptide antagonist of CXCR4 and Diprotin A (Ile-Pro-Ile, Peptides International, Louisville, Ky.) is a commonly used example of a CD26 inhibitor. This figure shows a consistent increase in MSC migration with the administration of a CD26 inhibitor. Thus, the invention provides for the use of a CD26 inhibitor for the treatment of renal organ dysfunction (i.e., damage or injury), particularly, in combination with the use of MSCs. For example, it is believed that inhibition of SDF-1 inactivation will increase the homing of administered MSCs to an injured kidney and potentiate renal repair.

### Example I

Bone marrow-derived MSCs from Fisher 344 rats were used for in vitro experiments. First, using FACS, it was demonstrated that ˜35% of MSCs express both CD26 and CXCR4 proteins on their surface. In addition, cultured MSCs express functional CD26 and can effectively inactivate SDF-1. Finally, MSC migration towards SDF-1 in a transwell culture system can be significantly increased by pre-incubation of MSCs with Diprotin A, a CD26 inhibitor, or decreased by pre-incubation with AMD 3100, a CXCR4 inhibitor. As a result of the in vitro data, it is reasoned that inhibiting CD26 will augment the SDF-1-CXCR4 mediated chemotactic activity of MSCs in vivo. Thus, the invention relates to pre-treatment or co-administration of MSCs with a CD26 inhibitor to augment the homing and renal protective functions of MSCs administered to a subject to treat a disease involving organ failure or organ dysfunction.

### Example II

All procedures involving animals are approved by the respective Institutional Animal Care and Use Committees of the University of Utah, Veterans Affairs Medical Center (Salt Lake City, Utah), Indiana University (Indianapolis, Ind.), and the University of Hamburg (Hamburg, Germany). Animals are housed at a constant temperature and humidity, with a 12:12-h light-dark cycle, and have unrestricted access to a standard diet and tap water. Adult male Sprague-Dawley (SD) and Fisher 344 (F344) rats weighing 200-300 g are preferably used (Charles River, Wilmington, Mass.).

I/R ARF is induced in isoflurane-anesthetized animals, and rectal temperature is maintained at 37° C. After a midabdominal laparatomy, kidneys are exposed and renal pedicles are clamped with atraumatic vascular clamps for about 40 min. While the clamps are applied, the left carotid artery is cannulated with PE-50 tubing for intra-aortic cell delivery immediately after reflow. A CD26 inhibitor, such as Diprotin A, or vehicle only (control medium) is administered 30 minutes prior to reflow. Administration of cells is performed either immediately, 30 minutes, or 24 h after reflow or surgery. At the appropriate time after visual confirmation of reflow, ≈10⁶ labeled MSC/animal in 0.2 ml SFM are given via the left carotid artery. Additional control ARF animals may also be used, wherein the control animals are treated identically but infused with 0.2 ml SFM instead of cells. Alternatively or in combination, a control group of F344 rats with ARF may also be infused with ≈10⁶ syngeneic fibroblasts in 0.2 ml SFM, using an identical protocol. Delayed infusions (≈10⁶ labeled MSC in 0.2 ml SFM) are preferably conducted in isoflurane-anesthetized animals 30 minutes after reflow via the left carotid artery. Incisions are closed with 4-0 silk, and animals are allowed to recover.

Quantification of cell numbers. CFDA-labeled green fluorescing cells are examined and quantified in kidneys at 2, 24, 36, 48, 60, and/or 72 h after ARF and cell infusions. Nuclei are stained with either Hoechst 33342 or propidium iodide, and nuclei are counted in at least three high-power fields (HPF) per section based on a calibrated confocal micrometer measurement bar. After this, total cell numbers of the studied tissue sections are calculated based on the number of nuclei and surface area of the section.

Based on the in vitro studies, it is believed that administration of MSCs following administration of the CD26 inhibitor Diprotin A, or co-administration of one or more CD26 inhibitor and MSCs, will result in increased migration of MSCs to the kidney and to significantly improved renal function in test animals by at least days 2 and/or 3, relative to MSC only treated test animals. Renal function may be assessed by serum creatinine and/or blood urea nitrogen levels. At 30 minutes postischemia, renal function is expected to be identically, or nearly identically, decreased in all treatment groups. However, subsequent to MSC administration renal function is expected to be significantly improved in CD26 inhibitor and MSC treated animals compared with MSC treated only (control medium) animals.

All references, including publications, patents, and patent applications, cited herein are hereby incorporated by reference to the same extent as if each reference were individually and specifically indicated to be incorporated by reference and were set forth in its entirety herein.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the scope of the appended claims.

What is claimed is:

1. A method of treating multi-organ failure, acute renal failure, chronic renal failure, or kidney dysfunction in a subject, the method comprising:
administering a therapeutically effective amount of a CD26 inhibitor to a subject; and
co-administering a therapeutically effective amount of mesenchymal stem cells to the subject.

2. The method according to claim 1, comprising administering a CD26 inhibitor selected from the group consisting of Diprotin A, L-Val-L-boroPro, sulphostin, P-epi sulphostin, sulphostin desulfonate, Lys[Z(NO₂)]-thiazolidide, Lys[Z(NO₂)]-pyrrolidide, LAF237, MK-0431, Sitagliptin, vildagliptin, Saxagliptin, Allogliptin, a salt or ester thereof, and combinations thereof.

3. The method according to claim 1, comprising administering a therapeutically effective amount of the CD26 inhibitor Diprotin A or a chemically modified analog thereof.

4. The method according to claim 3, comprising administering a chemically modified analog of Diprotin A, wherein the chemical modifications are selected from the group consisting of: modifications to the N and/or C terminal ends of the peptide; changes to the side chain of the amino acid; modification of the a carbon; replacing one or more D residues with one or more L residues; and introduction of amide bond replacements.

5. The method according to claim 1, comprising administering a therapeutically effective amount of the CD26 inhibitor Sitagliptin or vildagliptin.

6. The method according to claim 1, comprising administering allogeneic mesenchymal stem cells.

7. The method according to claim 1, comprising administering autologous mesenchymal stem cells.

8. The method according to claim 1, comprising treating renal failure associated with diabetes.

9. The method according to claim 1, comprising treating organ failure associated with a myocardial infarction.

10. The method according to claim 1, comprising treating acute renal failure in the subject.

11. The method according to claim 1, further comprising co-administering a chemotropic agent to the subject.

12. The method according to claim 11, comprising administering. SDF-1 or SDF-1 analogue as the chemotropic agent.

13. The method according to claim 1, comprising co-administering mesenchymal stem cells that express CD34 and CXCR4.

14. A method of treating multi-organ failure, acute renal failure, chronic renal failure, or kidney dysfunction in a subject, the method comprising:
treating mesenchymal stem cells in vitro with an effective amount of a CD26 inhibitor; and
administering a therapeutically effective amount of the CD26 treated mesenchymal stem cells to a subject.

15. The method according to claim 14, comprising administering a CD26 inhibitor selected from the group consisting of Diprotin A, L-Val-L-boroPro, sulphostin, P-epi sulphostin, sulphostin desulfonate, Lys[Z(NO2)]-thiazolidide, Lys[Z(NO2)]-pyrrolidide, LAF237, MK-0431, Sitagliptin, vildagliptin, Saxagliptin, Allogliptin, a salt or ester thereof, and combinations thereof.

16. The method according to claim 14, comprising administering Diprotin A or a chemically modified analog thereof.

17. The method according to claim 14, comprising administering a therapeutically effective amount of the CD26 inhibitor Sitagliptin or vildagliptin.

18. The method according to claim 14, further comprising co-administering a chemotropic agent to the subject.

19. The method according to claim 18, comprising administering SDF-1 or SDF-1 analogue as the chemotropic agent.

20.-24. (canceled)
